(19) United States
(12) Patent Application Publication
(10) Pub. No.: US 2009/0131352 A1
(43) Pub. Date: May 21, 2009

(54) Aptamers comprising arabinose modified nucleotides

(76) Inventors: Masad J. Dahma, St. Hubert (CA); Chang Geng Peng, Montreal (CA)

Correspondence Address: Pepper Hamilton LLP, 400 Berwyn Park, 899 Cassatt Road, Berwyn, PA, 19312-1183, US

(21) Appl. No.: 12/089,382

(22) PCT Filed: Oct. 3, 2006
(86) PCT No.: PCT/CA06/01635

§ 371 Date: Sep. 2, 2008

Related U.S. Application Data
(60) Provisional application No. 60/722,973, filed on Oct. 4, 2005.

(51) Int. Cl. A61K 31/711; C07H 21/04; A61P 31/18

(52) U.S. Cl. 514/44; 536/23.1

(57) ABSTRACT

Nucleic acid ligands (or aptamers) that form a G-tetrad containing at least one arabinose modified nucleotide are provided. Preferably, the arabinose modified nucleotide is 2′-deoxy-2′-fluoroarabinonucleotide (FANA) nucleotide. Methods of using aptamers the aptamers of the claimed invention are also provided.

### FIELD OF THE INVENTION

The invention relates generally to aptamers and more specifically to aptamers containing at least one arabinose modified nucleotide.

### BACKGROUND OF THE INVENTION

Oligonucleotide-based therapeutics have enormous potential for targeted therapy of cancer as well as inflammatory and infectious disease, exhibiting greater specificity and less toxicity than conventional chemotherapeutic drugs. The so-called “antisense” (AON) and “small interfering RNA” (siRNA) are the most prominent members of this class of agents [Stull, R. A. and Szoka, F. C. (1995) Pharmaceutical Research, 12: 465-483; Uhlmann E. and Peyman, A. (1990) Chemical Reviews, 90: 544-584.; Mittal, V. (2004) Nature Rev., 5: 355-365]. Aptamers and immunostimulatory oligonucleotides are the most recent additions to the large number of nucleic acid molecules being pursued as potential therapeutic agents. AONs and siRNAs are designed to target a specific mRNA, whereas aptamers and immunostimulatory oligonucleotides generally function by specific protein targets or activating a wide array of immune effector cells [Nimjee, S. M. et al. (2005) Annu. Rev. Med. 56: 555-83; Uhlmann E. and Vollmer J. (2003) Current Opinion in Drug Discovery & Development 6: 204-217].

Excellent progress towards clinical applications of aptamers has been made [Hicke, B. J. et al. (1996) J. Clin. Investig. 98: 2688-2692; Pietras, K. et al. (2002) Cancer Res. 62: 5476-5484; White, R. R. et al. (2003) Proc. Natl. Acad. Sci. U.S.A. 100: 5028-5033)]. Aptamers have gained acceptance with the recent FDA approval of Macugen®, a sugar-modified RNA analog (2° Fribose, 2′-O-methylribose, 3′-pegylated aptamer, M.Wt. 50 kD) indicated for the treatment of neovascular age-related macular degeneration (AMD) [(a) Eyetech Study Group (2002) Preclinical and phase 1A clinical evaluation of an anti-VEGF pegylated aptamer (EYE001) for the treatment of exudative age-related macular degeneration, Retina, 22: 143-52; (b) Eyetech Study Group (2003) Antivascular endothelial growth factor therapy for subfoveal choroidal neovascularization secondary to age-related macular degeneration: phase II study results, Opthalmology, 110: 979-86]. Nucleic acid aptamers have also been shown to control viral gene expression, including HIV, in vitro [(a) Sullenger B. A., Gallardo H. F., Ungers G. E. and Gilboa E. (1991) Analysis of trans-acting response decoy RNA-mediated inhibition of human immunodeficiency virus type 1 transactivation, Journal of Virology, 65: 6811-6816; (b) Zimmermann K., Weber S., Dobrovnik M., Hauber J. and Bohnlein E. (1992) Expression of chimeric neo-rev response element sequences interferes with rev-dependent HIV-1 gag expression, Human Gene Therapy, 3: 155-161; (c) Lee T. C., Gallardo H. F., Ungers G. E. and Gilboa E. (1992) Overexpression of RRE-derived sequences inhibits HIV-1 replication in CEM cells. New Biologist, 4: 66-74]. Aptamers may also prove useful for the treatment of other important human maladies, including infectious diseases, cancer, and cardiovascular disease. A common technique by which oligonucleotide aptamers are obtained relies on the systematic evolution of ligands by exponential enrichment (SELEX) process [Tuerk, C. and Gold, L. (1990) Science, 249, 505-510); Ellington, A. D. and Szostak, J. W. (1990) Nature, 346: 818-822]. The resulting oligonucleotides are more commonly referred to as “aptamers”, derived from the Latin word “aptus”, meaning “to fit”. These single- or double-stranded molecules are typically capable of binding proteins and, as such, serve as “sinks” by blocking the protein from further function (Baltimore D. (1988) Nature 335: 395-3961.

The utility of nucleic acid aptamers in vivo and their possible application in pharmacotherapy, as with other oligonucleotide-based therapies, face some key hurdles e.g., delivery, cellular uptake and biostability. There is a need to develop chemical modifications to produce clinically useful molecules. Initial work with oligodeoxynucleotides (DNA) was undertaken with unmodified, natural molecules. It soon became clear however, that native DNA was subject to relatively rapid degradation, primarily through the action of 3′ exonucleases, but as a result of endonuclease attack as well. oligoribonucleotides (RNA) are subject to the same considerations and are, in fact, generally more susceptible to nuclease degradation. The same issues apply to aptamers where nuclease stability is highly desirable. Given that the protein binding activity of aptamers is strongly dependent on the folding of the oligonucleotide structure (3D structure), it is highly desirable that such structure is of high thermal stability.

Until now, several methods have been devised to improve the stability of aptamers, most of which make use of SELEX. Nolte et al. have reported a mirror-design RNA aptamer (or “Spiegelmers”), which consists of selecting a normal RNA aptamer (D-RNA) against the enantiomer of a target protein, the mirror image of the target protein (D-amino acids), by using standard SELEX. When the resulting RNA aptamer (D-RNA) is converted to its enantiomeric form, L-RNA, with the same base composition, the L-RNA exhibits high binding affinity to the native protein molecule (L-amino acids) and high resistance against cleavage by nucleases. This strategy is limited to cases where an enantiomer of the target molecule is available [Nolte, A. et al. (1996) Nat. Biotechnol., 14: 1116-1119]. Another method for the stabilizing RNA aptamers consists of a chemical modification after the RNA molecules have been selected by SELEX. Normally such modifications are introduced by incorporation of 2′-O-methylribonucleotides into the native RNA structure. However, this strategy causes structural changes of the RNA molecules and often results in loss of RNA aptamer activity [Lebruska, L. L. and Maher, L. J. (1999) Biochemistry, 38: 3168-3174]. A variation of the SELEX method generates nuclease resistant RNA molecules by employing modified nucleoside triphosphates instead of the natural substrates (dNTPs or rNTPs) [U.S. Pat. No. 5,660,985, both entitled “High Affinity Nucleic Acid Ligands Containing Modified Nucleotides”, and U.S. Pat. No. 6,387,620, entitled “Transcription-free SELEX”]. However, some of these chemistries are incompatible with SELEX as the monomeric 5′-triphosphates units are not substrates of DNA/RNA polymerases. Thus far, 2′-modified-2′-deoxynucleoside 5′-triphosphates [Pagratis, N. C., et al. (1997) Nat. Biotechnol., 15: 68-73], nucleoside 5′-(alpha-P-borano)triphosphates [Lato, S. M. (2002) Nucleic Acids Res., 30: 1401-1407], nucleoside 5′-(alpha-thio)triphosphates (Jhaveri, S. et al. (1998) Bioorg. Med. Chem. Lett., 8: 2285-2290], and more recently, 4′-thioribonucleoside 5′-triphosphates [Kato, Y. et al. (2005) Nucleic Acids Res. 33: 2942-2951] are the most used triphosphates for SELEX. Among these, 2′-modified rNTPs, 2′-fluoro-2′-deoxy-ribopyrimidine (2′F-RNA) and 2′-amino-2′-deoxy-ribopyrimidine (2′-NH₂-RNA) nucleoside triphosphates are frequently used. A number of nuclease-resistant RNAs, including the vascular endothelial growth factor-binding aptamer, Macugen, were isolated using primarily 2′F-rU/rC and 2′-NH₂-rU/rC 5′-triphosphates [Ruckman, J. (1998) J. Biol. Chem. 273: 20556-20567].

A DNA aptamer targeted toward thrombin, a key protease involved in the blood clotting cascade, has been identified and related studies have been performed. This aptamer, first identified via SELEX, consists of a 15-nt sequence containing six thymidine (dT) and nine deoxyguanosine (dG) nucleotides, namely 5′-dGGTTGGTGTGGTTGG-3′. Under certain conditions, this oligonucleotide is known to fold into a quadruplex structure, which contains two G-quartets and three lateral loops, usually referred to as a “chair structure” (FIG. 1) [Bock, L. C. et al. (1992) Nature 355: 564]. Each quartet adopts a square planar configuration, each dG residue interacting with the adjacent one via two hydrogen bonds and behaving as both H-bond acceptor and donor. Potassium ions stabilize the entire structure by coordination to the guanine bases. A similar folding was reported on the crystal structure of the aptamer-thrombin complex [Padmanabhan, K. et al. (1993) J. Biol. Chem. 268: 17651]. There are several other examples of G-quadruplex structures reported in the literature, some of which are being tested as therapeutic agents themselves, specifically as antivirals and anticancer agents [see Saccà, B. et al. (2005) Nucleic Acids Res. 33: 1182-119, and references therein].

Several studies aimed at modifying this aptamer have been reported, but very few, if any, have led to an improvement over the original molecule. For example, Heckel and Mayer reported that the introduction of thymines modified with a nitrophenylpropyl moiety (T-NPP) at certain positions generally abolished interaction of the aptamer with thrombin [Heckel, A. and Mayer, G. (2005) J. Am. Chem. Soc. 127: 822-823]. Di Giusto and King reported the synthesis of circular aptamers targeted against thrombin with improved nuclease resistance and anticoagulant activity compared to those of the canonical thrombin DNA aptamer [Di Giusto, D. A. and King, G. C. (2004) J. Biol. Chem. 279: 46483-46489]. However, circularization of the aptamers produces a mixture of constructs and requires a ligase enzyme, making the method very difficult to scale-up. Other attempts to circularize the thrombin-binding DNA aptamer via chemical methods abolished the anti-thrombin activity [Buijsman, R. C. et al. (1997) Bioorg. Med. Chem. Letters 7: 2027-2032]. Recently, Seela and coworkers reported the insertion of a hairpin-forming sequence GCGAAG into the position of the central loop of the thrombin-binding aptamer. This construct was able to form both a G-quartet and a joined minihairpin structure. According to the Tₘ data, the minihairpin induces a structural change in the aptamer section. Binding to thrombin was not investigated [Rosemeyer, H. et al. (2004) Helvetica Chimica Acta 87: 536-522]. Saccà et al. studied the effect of backbone charge and atom size, base substitutions as well as the effect of modification at the sugar 2′-position as analyzed by spectroscopy. All sugar (ribose, 2′-O-methylribose) and phosphate(methylphosphonate, phosphorothioate) led to a reduction in the thermal stability of the aptamer [Sacca, B. et al. (2005) Nucleic Acids Res. 33: 1182-1192]. In fact, the 2′-O-methylribose modification led not only to a destabilization of the structure, but also to a complete changing of the G-quartet conformation. As such, the structure of the thrombin aptamer is particularly sensitive to chemical modifications. Furthermore, previous studies have shown that replacing the native DNA bases by modified bases generally disrupt the aptamer structure.

The phosphorothioate octanucleotide dTTGGGGTT [PS-dT₂G₄T₂] is a compound that binds to the viral envelope protein gp120 of the human immunodeficiency virus (HIV), preventing fusion of HIV to the cellular CD4 receptor [Wyatt J. R. et al. (1994) Proc. Nat. Acad. Sci. USA 90: 1356-1360]. PS-dT₂G₄T₂ forms a parallel-stranded tetramer stabilized by G-quartets (G-tetrads). Wyatt et al. [Proc. Nat. Acad. Sci. USA 90: 1356-1360] also showed that its G-tetrad structure and certain phosphorothioate linkages were necessary for inhibition of viral infection [Stoddart, C. A. et al. (1998) Antimicrob Agents Chemother. 42: 2113-2115].

The oligomer dGGGGTTTTGGGG is derived from the telomere d(T₄G₄) repeat of Oxytricha [Smith, F. W. and Feigon, J. (1992) Nature, 356: 164-168). NMR studies showed that this compound, like the antithrombin aptamer, forms a G-quartet structure [Smith, F. W. and Feigon, J. (1992) Nature, 356: 164-168; Smith F. W. and Feigon J. (1993) Biochemistry 32: 8682]. As G-tetrads are found in human telomeres, they are of particular interest for anticancer drug discovery efforts. These G-tetrad structures may be used to inhibit telomere extension (by inhibiting telomerase), a process that occurs selectively in cancer cells [Kerwin, M. (2000) Current Pharmaceutical Design 6: 441-471].

The anti-thrombin oligomer dGGTTGGTGTGGTTGG displays a characteristic circular dichroism (CD) spectrum, referred to as a “Type II” CD spectrum. A type II CD profile is indicative of a unimolecular G-quartet in which two of the guanine residues are in the anti conformation, and the two others in the syn conformation [Macaya, R. F. et al. (1993) Proc. Natl. Acad. Sci. U.S.A., 90: 3745-3749]. The term G (anti) refers to a guanosine nucleoside structure in which the guanine base is oriented away from the sugar ring to which is attached, whereas in the G (syn) conformation the guanine base is placed directly above the sugar ring structure. The anti and syn conformational change comes about the rotation of the Cl′-N9 glycosidic bond [W. Saenger, in “Principles of Nucleic Acids Structure”, C. R. Cantor (editor); Springer-Verlag, 1983]. The “Type II” CD spectrum display a positive band at ˜295 nm and a negative band at ˜260 nm. On the other hand, a “Type I” CD spectrum displays a positive CD band at ˜265 nm and a negative band at ˜240 nm that correlates with a intermolecular G-tetrad with only G (anti) residues [Williamson, J. R. (1994) G-Quartet Structures in Telomeric DNA. Annu. Rev. Biophys. Biomol. Struct. 23: 703-730]. Thus these two types of CD spectra strongly correlate to the conformation of the G-quartet core.

The telomeric dGGGGTTTTGGGG sequence, like the anti-thrombin sequence described above, exhibits a Type II CD spectrum, consistent with a G-tetrad with guanines in both syn and anti conformations (Lu, M. et al. (1993) Biochemistry, 32: 598-601; Smith, F. W. and Feigon, J. (1992) Nature, 356: 164-168). By contrast, the sequence dTTGGGGTT (either with phosphodiester linkage (PO) or phosphothioate linkage (PS)) shows a Type I CD spectrum, resulting from a G-quadruplex structure in which all guanine bases adopt the anti conformation (Wyatt, J. R. et al. (1994) Proc. Natl. Acad. Sci. U.S.A., 91: 1356-1360).

There is a need in the art to improve the nuclease stability of aptamers generally including, in particular, those that are capable of forming a G-tetrad such as those described above. Furthermore, it is preferable that such modification does not significantly decrease the subtle binding interaction of the selected native aptamer.

### SUMMARY OF THE INVENTION

According to one broad aspect of the invention, nucleic acid ligands (or aptamers) capable of forming a G-tetrad and comprising at least one arabinose modified nucleotide are provided. Preferably, the arabinose modified nucleotide is 2′-deoxy-2′-fluoroarabinonucleotide (FANA). The arabinose modified nucleotide is preferably in the loop of the G-Tetrad or alternatively a guanosine residue of the G-tetrad.

In a preferred embodiment of the invention the aptamer is an antithrombin aptamer, preferably having the sequence: dGGTTGGTGTGGTTGG (15-nt).

In another preferred embodiment the aptamer is an anti-HIV aptamer, preferably having the sequence: dT₂G₄T₂ (8-nt).

In another preferred embodiment of the invention the aptamer comprises a dG₄T₄ repeat, preferably dG₄T₄G₄ (12-nt), dG₄T₄G₄T₄G₄ (20-nt), and dG₄T₄G₄T₄G₄T₄G₄ (28-nt).

In specific embodiments of the invention, the aptamer has a sequence according any one of SEQ ID NOS. 1-3, 4-14, 19-24 and 26-28.

In specific embodiments, the aptamer may have any number of arabinonucleotides at any location in the aptamer, for example:

5′-ADADADADADADADA-3′
5′-AADADDADDDAADAD-3′
5′-AAAADAAADADDDAD-3′
etc.

wherein A is an arabinonucleotide and D is a 2′-deoxyribonucleotide.

In other embodiments of the invention, the aptamer is fully substituted with arabinonucleotides. For example:

5′-AAAAAAAAAAAAAAA-3′

In a preferred embodiment of the present invention, chimeras constructed from 2′-deoxyribonucleotide (DNA) and 2′-deoxy-2′-fluoroarabinonucleotide (FANA) capable of binding thrombin selectively are provided.

In other embodiments of the invention, an aptamer of any one of sequence 5′-GGTTGGTGTGGTTGG-3′, dT₂G₄T₂ and d[G₄T₄G₄]ₙ is provided having a sugar-phosphate backbone composition selected from any combination of arabinose and deoxyribose nucleotides. Preferably, the arabinose nucleotides are 2′-deoxy-2′-fluoroarabinonucleotide (FANA).

In other embodiments of the invention, the arabinonucleotide comprises a 2′ substituent selected from the group consisting of fluorine, hydroxyl, amino, azido, alkyl, alkoxy, and alkoxyalkyl groups. In a further embodiment of the invention, the alkyl group is selected from the group consisting of methyl, ethyl, propyl, butyl, and functionalized alkyl groups such as ethylamino, propylamino and butylamino groups. In embodiments, the alkoxy group is selected from the group consisting of methoxy, ethoxy, proproxy and functionalized alkoxy groups such as —O(CH₂)_{q}—R, where q=2-4 and —R is a —NH₂, —OCH₃, or —OCH₂CH₃ group. In embodiments, the alkoxyalkyl group is selected from the group consisting of methoxyethyl, and ethoxyethyl. In embodiments, the 2′ substituent is fluorine and the arabinonucleotide is a 2′-fluoroarabinonucleotide (FANA). Preferably, the FANA nucleotide is araF-G and araF-T.

In other embodiments of the invention, the aptamer comprises one or more internucleotide linkages selected from the group consisting of:

a) phosphodiester;

b) phosphotriester;

c) phosphorothioate;

d) methylphosphonate;

e) boranophosphate and

f) any combination of (a) to (e).

According to another broad aspect of the invention, a method for increasing at least one of nuclease stability or selective binding of an aptamer is provided. The method comprises replacing at least one nucleotide of the aptamer, preferably in a loop of an aptamer that forms a G-tetrad, with an arabinose modified nucleotide, preferably 2′-deoxy-2′-fluoroarabinonucleotide (FANA).

According to another broad aspect of the invention a pharmaceutical composition is provided, comprising the aptamer of the present invention along with a pharmaceutically acceptable carrier.

According to another broad aspect of the invention, a use of an aptamer of the present invention is provided for the preparation of a medicament for inhibiting thrombin.

According to another broad aspect of the invention, a use of an aptamer of the present invention is provided for the preparation of a medicament for treating or preventing HIV infection.

According to another broad aspect of the invention, a use of an aptamer of the present invention is provided for the preparation of a medicament for treating or preventing cancer.

According to another broad aspect of the invention, a method of inhibiting thrombin or preventing or treating HIV or cancer in a patient in need thereof is provided. The method comprises administering to the patient a therapeutically effective amount of the pharmaceutical composition of the invention.

According to another broad aspect of the invention a commercial package is provided. The commercial package comprises the pharmaceutical composition of the present invention together with instructions for its use.

### BRIEF DESCRIPTIONS OF THE DRAWINGS

The invention will now be described in greater detail having regard to the appended drawings in which:

FIG. 1 illustrates the guanine quadruplex (G-quartet) (Left) and diagram of the intramolecular G-quartet of the thrombin binding DNA aptamer (Right). The structure of FANA units (thymine and guanine base) is also shown (bottom left).

FIGS. 2a and 2b illustrates thermal melting profiles measured at 295 nm in buffer a) 10 mM Tris, pH 6.8; b) 10 mM Tris, pH 6.8, 25 mM KCl, at a final strand concentration of 8 μM. The Tₘ (melting temperature) data is provided in Table 1.

FIG. 3 illustrates Tₘ versus concentration dependence study carried out in a buffer consisting of 10 mM Tris, pH 6.8, 25 mM KCl.

FIG. 4 illustrates heating and cooling Tₘ transitions of G-tetrads formed by arabinose modified oligonucleotides and control DNA oligonucleotide in a buffer of 10 mM Tris, 25 mM KCl, pH 6.8, at a final strand concentration of 8 μM.

FIG. 5 illustrates CD spectra of oligonucleotides in a buffer consisting of 10 mM Tris, pH 6.8 without/with 25 mM KCl at 15° C. at a final strand concentration of 8 μM.

FIG. 6a illustrates stability of aptamers to 10% fetal bovine serum (FBS) as monitored by polyacrylamide gel electrophoresis (time points: 0, 0.25, 0.5, 1, 2, 6, 24 h).

FIGS. 6b and 6c illustrates stability curve of aptamers to 10% FBS as monitored by polyacrylamide gel electrophoresis.

FIGS. 7a and 7b illustrates nitrocellulose filter binding curves for aptamers following exposure to bovine thrombin.

FIG. 8 illustrates CD spectra of dT₂G₄T₂ and related sequences (PG17, 28, 19, 20, 21, 22, 23 & 24) obtained in phosphate buffered saline (PBS buffer), 137 mM NaCl, 2.7 mM KCl, 1.5 mM KH₂PO₄, 8 mM Na₂HPO₄, pH 7.2 at 25° C.; strand concentration: 20 μM.

FIG. 9 illustrates temperature-dependent CD spectra for PG17, PG18, PG19 and PG20 and resulting T, curves for each complex. The solvent is phosphate buffered saline (PBS buffer), 137 mM NaCl, 2.7 mM KCl, 1.5 mM KH₂PO₄, 8 mM Na₂HPO₄, pH 7.2 at 25° C.; strand concentration: 20 μM; 10 min equilibrium time was set at each temperature studied. The resulting Tₘ curves were generated by plotting the maximum absorbance at 265 nm wavelength (normalized) vs temperature; the corresponding Tₘ data are shown in Table 2.

FIG. 10. Temperature-dependent CD spectra for PG24: phosphate buffered saline (PBS buffer), 137 mM NaCl, 2.7 mM KCl, 1.5 mM KH₂PO₄, 8 mM Na₂HPO₄, pH 7.2 at 25° C.; strand concentration: 20 μM; solutions were allowed to equilibrate for 10 min after each temperature change.

FIG. 11. CD spectra of dT₄G₄T₄ and related sequences (PG25-28): 10 mM sodium phosphate buffer, 0.1 mM EDTA, pH 7 and 200 mM NaCl; strand concentration: 10 μM.

FIG. 12a-e illustrates a: Tₘ profile of dT₄G₄T₄ and related sequences (PG25-28), whereas FIG. 12b-e: illustrate the effect of heating and cooling process during thermal melting measurements at 10 mM sodium phosphate buffer, 0.1 mM EDTA, pH 7 and 200 mM NaCl; strand concentration: 100 μM.

### DETAILED DESCRIPTION

This invention relates to modified oligonucleotides that are capable of selectively binding to a protein target. In particular, aptamers having short strands of DNA and modified arabinonucleic acids is shown, in contrast to the common methods described above, which have concentrated on the use of linkers, hairpins and modified nucleoside derived from the naturally occurring units (i.e., DNA and RNA nucleotides).

This invention encompasses the characterization of a series of sugar modified nucleic acid ligands that bind thrombin. These nucleic acid ligands (or aptamers) contain arabinose modified nucleotides conferring improved characteristics on the ligand, such as improved folding (thermal stability, Tₘ) and stability against nucleases present in body fluid. The invention also encompasses the induction and stabilization of G-tetrads comprising arabinose sugars. Preferably, the sugar modified nucleotides are 2′-deoxy-2′-fluoroarabinonucleotides (FANA). The method for generating the FANA modified ligand necessitates the substitution of DNA bases in a known anti-thrombin aptamer dGGTTGGTGTGGTTGG (15-nt), anti-HIV aptamer dTTGGGGTT (8-nt) and telomeric oligonucleotide dGGGGTTTTGGGG (12-nt), for FANA residues. In all cases, folding was assayed using circular dichroism and UV melting experiments, whereas for dGGTTGGTGTGGTTGG, thrombin binding was determined using a nitrocellulose filter binding assay. Selective, specific and efficient binding of such FANA modified nucleic acid ligands to thrombin is demonstrated. Previous studies with the anti-thrombin aptamer dGGTTGGTGTGGTTGG have shown that replacing the native DNA bases by modified bases generally disrupts the aptamer structure. The compounds disclosed here represent the first examples of FANA modified aptamers that bind thrombin effectively.

This invention provides FANA nucleotides that are compatible with the structure and activity of the thrombin binding DNA aptamer; in addition, it is shown that the FANA modification can be effected without SELEX; rather, it involves incorporation of a sufficient number of FANA units (via solid-phase chemical methods) to allow increased nuclease and thermal stability without significant loss of binding affinity for the target biomolecule. Unexpectedly, in some cases, target binding activity is improved over the known thrombin binding DNA aptamer.

The thermal stability of G-quadruplexes (G-tetrads) is also shown to be improved by inserting FANA residues within the oligonucleotide chain of the thrombin binding aptamer 5′-dGGTTGGTGTGGTTGG, the anti-HIV aptamer dT₂G₄T₂ and the telomeric oligonucleotide dG₄T₄G₄. Accordingly, 2′-deoxy-2′-fluoro-β-D-arabinoguanosine (araF-G) alone, or in combination with deoxyguanosine units, are capable of folding into a G-quartet structure. Based on these findings, the araF-G alone, or in combination with deoxyguanosine (dG) can be employed for stabilizing other G-quartet structures, including those of therapeutic interest described above, thereby enhancing their properties in vivo. As such, according to another broad aspect of the invention, FANA-DNA oligonucleotide chimeras of G-quartet containing aptamers are provided.

A “therapeutically effective amount” refers to an amount effective, at dosages and for periods of time necessary, to achieve the desired therapeutic result. A therapeutically effective amount of a modified nucleic acid of the invention may vary according to factors such as the disease state, age, sex, and weight of the individual, and the ability of the modified nucleic acid to elicit a desired response in the individual. Dosage regimens may be adjusted to provide the optimum therapeutic response. A therapeutically effective amount is also one in which any toxic or detrimental effects of the compound are outweighed by the therapeutically beneficial effects. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, an oligonucleotide of the invention can be administered in a time release formulation, for example in a composition which includes a slow release polymer. The modified oligonucleotide can be prepared with carriers that will protect the modified oligonucleotide against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound, such as an oligonucleotide of the invention, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, an oligonucleotide of the invention may be formulated with one or more additional compounds that enhance its solubility.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

The following examples are illustrative of various aspects of the invention, and do not limit the broad aspects of the invention as disclosed herein.

### EXAMPLE 1

### Chemical Synthesis of Oligonucleotides

The sequence and composition of the oligomers prepared in this study are shown in Table 1 and Table 2. FANA modified aptamer syntheses were carried out on a 1 μmol scale on an Applied Biosystems (ABI) 3400A synthesizer using the standard β-cyanoethylphosphoramidite chemistry according to published protocols [E. Viazovkina, M. M. Mangos, M. I. Elzagheid, and M. J. Damha (2002) Current Protocols in Nucleic Acid Chemistry, Unit 4.15)]. The final concentrations of the monomers were 0.10 M for 2′-deoxyribonucleoside phosphoramidites and 0.125 M for the ara F phosphoramidites. The coupling time was extended to 150 seconds for the 2′-deoxyribonucleoside phosphoramidites (dC, dG), 15 min for the modified araF nucleosides. These conditions gave about 99% average coupling yields and usually over 100 optically density units (A260) in yield. Aptamers were purified by anion exchange HPLC and kept at −20° C. for further use.

### EXAMPLE 2

### UV Thermal Denaturation Studies of Thrombin Binding Aptamers

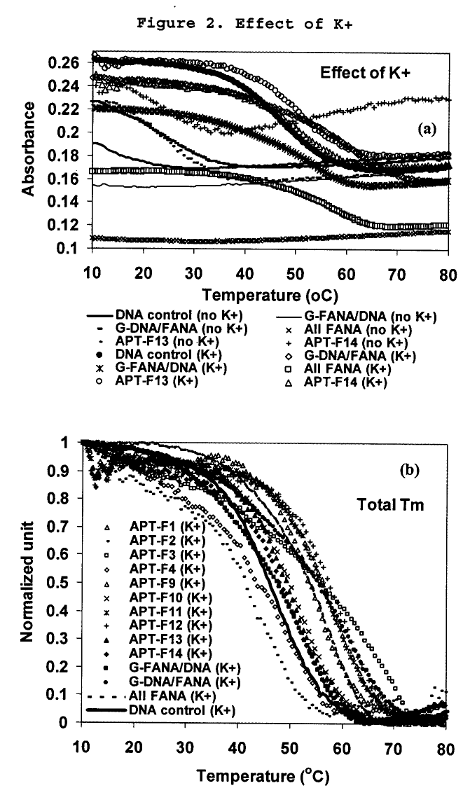

UV thermal denaturation data were obtained on a Varian CARY 1 spectrophotometer equipped with a Peltier temperature controller. Aptamers were dissolved in Tₘ buffer (10 mM Tris, pH 6.8 with and without 25 mM KCl) at a final concentration of 8 μM. Aptamers were annealed in Tₘ buffer at 80° C. for 10 minutes, naturally cooled down to room temperature and refrigerated (4° C.) overnight before measurements. The annealed samples were transferred to pre-chilled Hellma QS-1.000 (Cat #114) quartz celled, sealed with a Teflon-wrapped stopper and degassed by placing them in an ultrasound bath for 1 min. Extinction coefficients were obtained from the following internet site (http://paris.chem.yale.edu/extinct.html) and FANA modified aptamers were assume to have the same extinction coefficient as the regular DNA aptamer. Denaturation curves were acquired at 295 nm at a rate of heating of 0.5° C./min. The data were analyzed with the software provided by Varian Canada and converted to Microsoft Excel. Absorbance versus temperature profiles were adapted for a unimolecular transition. Slopping baselines were achieved by constructing liner least-squares lines for associated and dissociated parts and extrapolating to both ends of the melting curve. Consequently, a plot of the fractions of single strands in the G-quadruplex state (α) versus temperature was constructed and used to calculate the Tₘ value by interpolating to α=0.5 (FIGS. 2a and 2b).

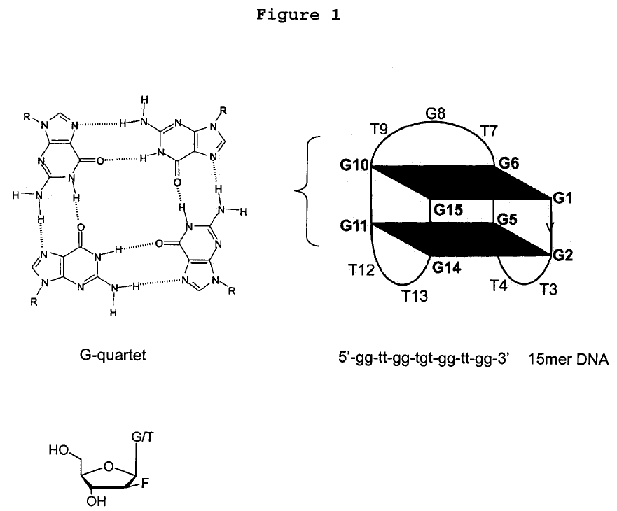

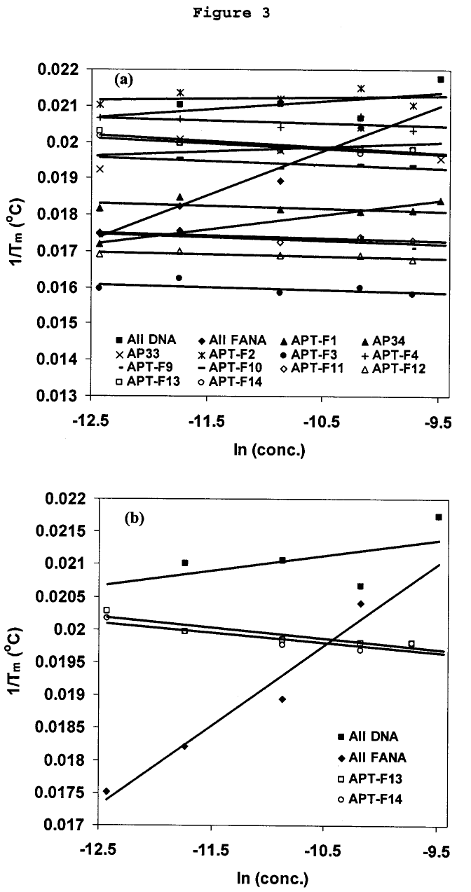

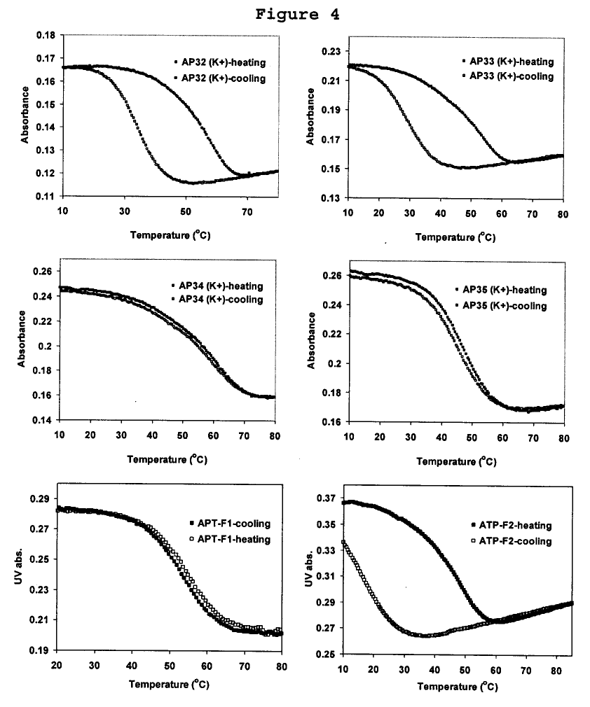

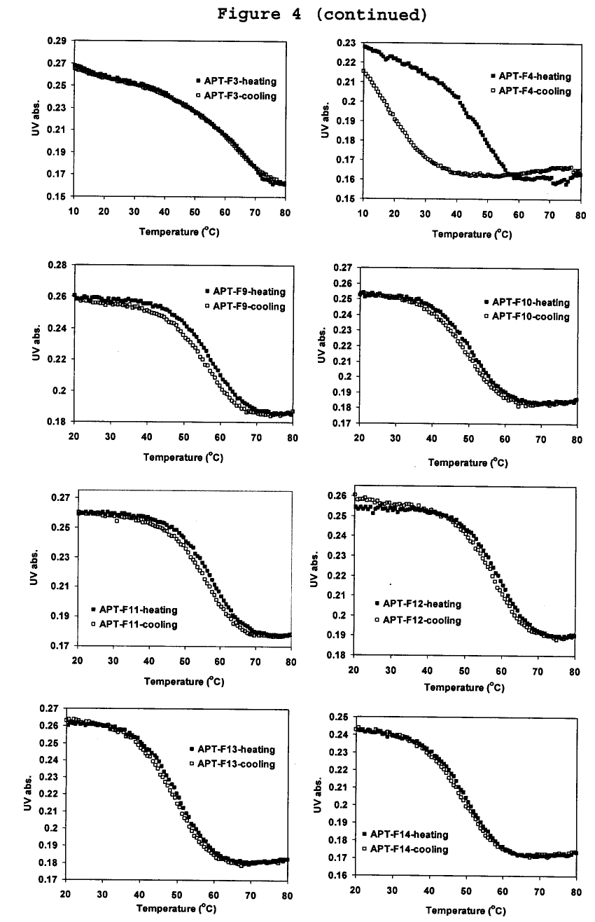

Tₘ concentration dependence studies were also conducted in the same way at 295 nm using aptamers with different concentrations ranging from 4 to 76 μM. Starna quartz cells (Starna Cells, Inc., Cat. # 1-Q-1) with 1 mm path length were used to reduce the amount of aptamers required (FIG. 3). The data shows that incorporation of FANA residues into the oligonucleotide backbone leads to an increase in the melting temperature of the complex formed (ΔTₘ up to +3° C./FANA modification). The structure is stabilized by potassium ion as shown in FIG. 2, consistent with the formation of a unimolecular G-tetrad structure (see Example 3). The unimolecularity of folding was ascertained for some of the sequences by measuring the Tₘ value at varying oligonucleotide concentrations (FIG. 3), and by obtaining heating and cooling Tₘ curves (FIG. 4). Unimolecular folding is characterized by a Tₘ value that is independent of oligonucleotide concentration, e.g. AP34, AP35, APT-13 and APT-F14, but not AP32 and AP33 (FIG. 3), and by fast kinetics of melting and re-annealing, e.g. AP34, AP35, APT-13 and APT-F14, but not AP32 and AP33 (FIG. 4). Note that all Type II aptamers (as assessed by CD; see Example 3) displayed identical heating and cooling Tₘ curves (FIG. 4), consistent with the fast kinetics of folding/unfolding that characterizes the unimolecular structure shown in FIG. 1.

### EXAMPLE 3

### Circular Dichroism (CD) Spectra of Thrombin Binding Aptamers

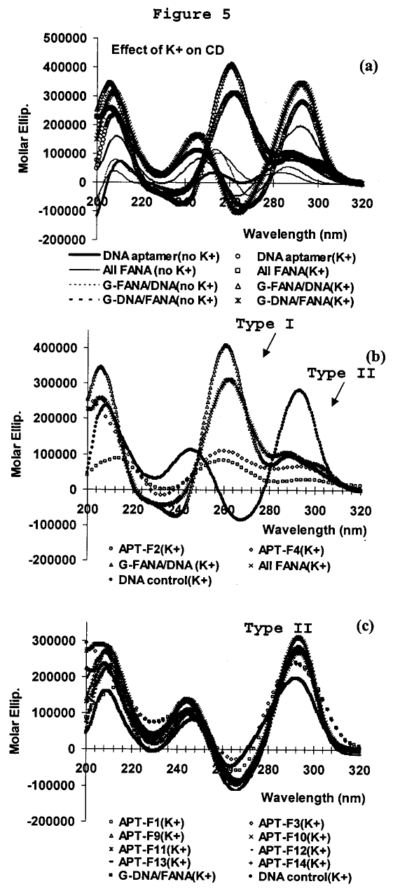

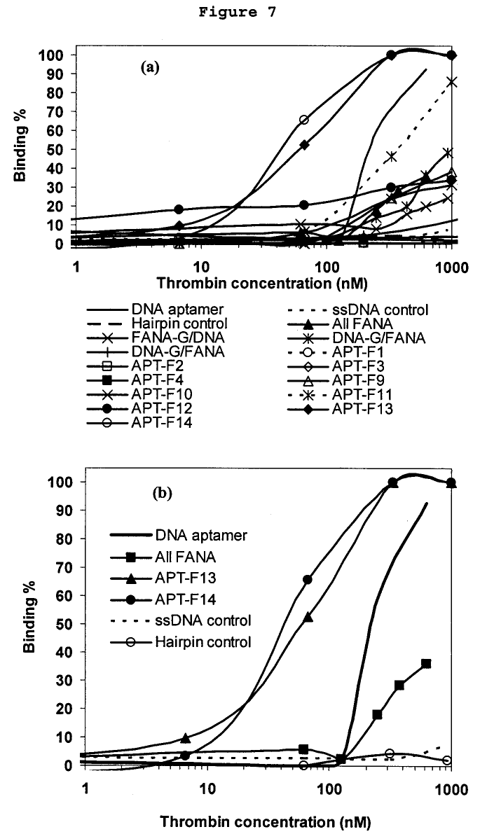

CD spectra (200-320 nm) were collected on a Jasco J-710 spectropolarimeter at a rate of 100 nm/min using fused quartz cells (Hellma, 165-QS). Measurements were carried out in Tₘ buffer (10 mM Tris, pH 6.8 with and without 25 mM KCl) at a concentration of 8 μM. Temperature was controlled by an internal circulating bath (VWR Scientific) at constant temperature (15° C.). The data were processed on a PC computer using J-700 Windows software supplied by the manufacturer (JASCO, Inc.). To facilitate comparisons, the CD spectra were background subtracted, smoothed and were corrected for concentration so that molar ellipticities could be obtained (FIGS. 5a-c). These experiments assess the impact of the FANA modification on the aptamer structures. CD spectra of aptamers revealed the formation of two different G-tetrad structures, referred to as “I” and “II” (Table 1 & FIG. 5). The “II” type CD signature corresponds to the well-characterized, potassium (K+) induced, G-tetrad structure adopted by the all-DNA aptamer dGGTTGGTGTGGTTGG. In fact, only type-II aptamers showed an affinity to the target protein (human thrombin), and not all type-II aptamers bound to thrombin (Table 1 & FIG. 7). For example, the type-II aptamers AP-F13 and AP-F14 were found to bind to human thrombin with a higher affinity relative to the all-DNA aptamer, whereas APT-F1 and F3 bound weakly, if at all to thrombin. Type II G-tetrad structures were also found to be very stable particularly when DNA-G (dG) residues with “anti” glycosidic bonds are replaced by FANA-G. The stabilization of G (anti) residues over G (syn) arises from the steric interactions the 2′-fluorine atom and the G base in the syn conformation. As a result FANA-G residues induce and stabilize Type II G-tetrads containing anti G residues. If dG (syn) positions are replaced by FANA-G residues, the aptamer switches to the type “I” G-tetrad, in which all guanines likely adopt the anti conformation (Table 1 and FIG. 5). In these cases, binding to thrombin was lost, consistent the exquisite specificity of thrombin to type-II DNA and type-II FANA-DNA structures. The same principles applied to the type-II telomeric oligonucleotide series derived from dGGGGTTTTGGGG (Example 6).

### EXAMPLE 4

### Nuclease Stability Assay

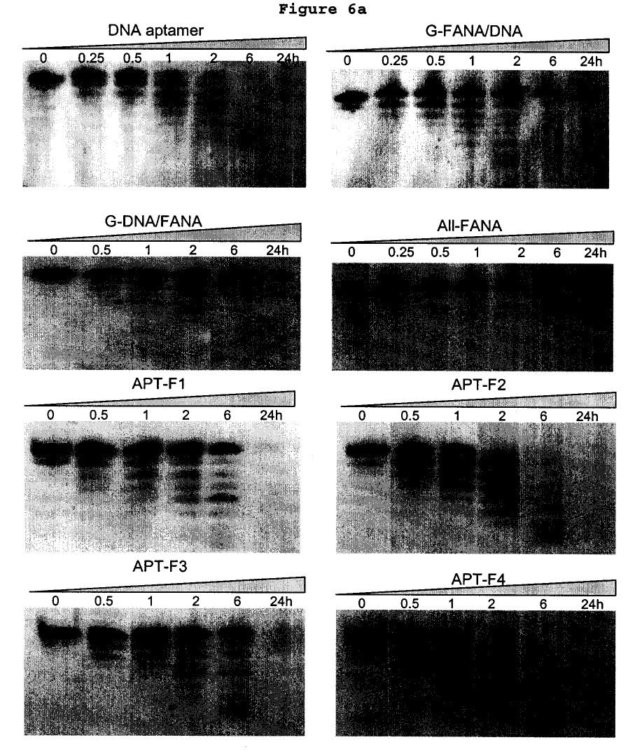

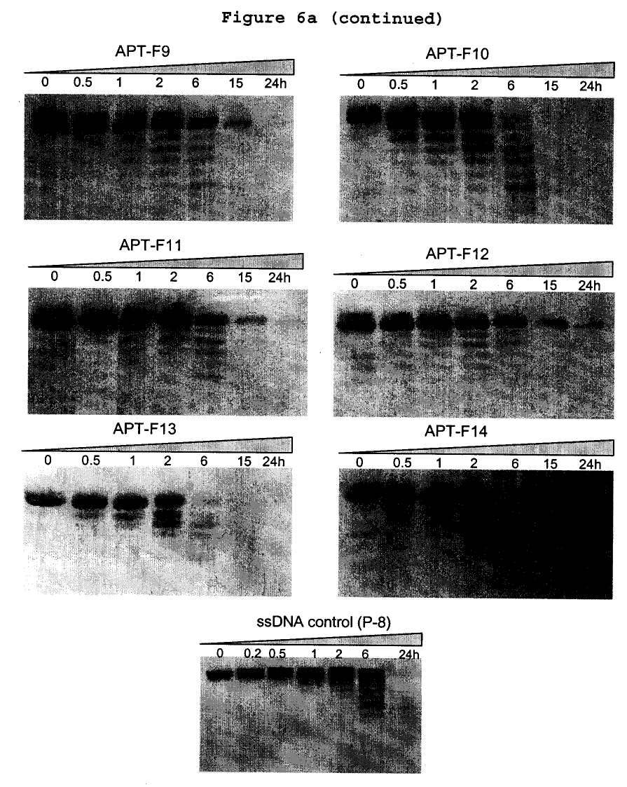

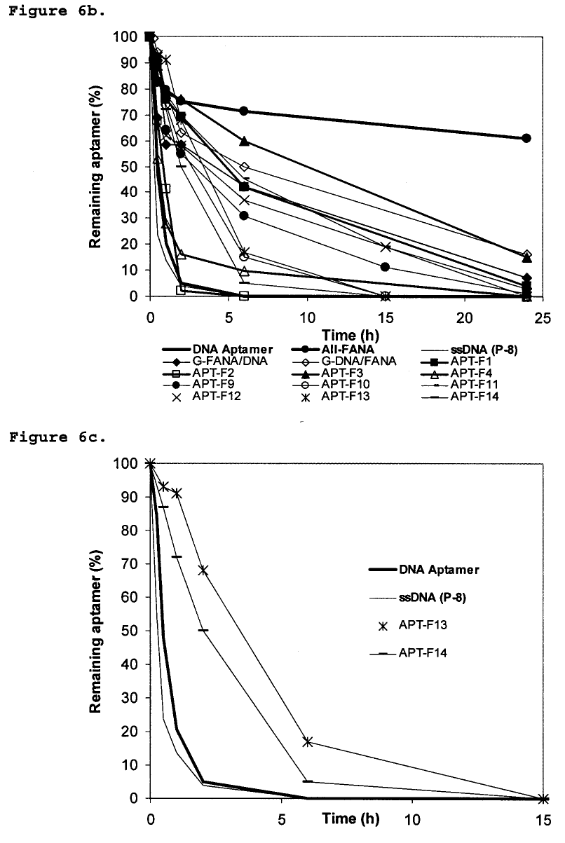

Nuclease stability of aptamers was conducted in 10% Fetal Bovine Serum (FBS, Wisent Inc., Cat. #080150) diluted with multicell Dulbeco's Modification Eagle's Medium (DMEM, Wisent Inc., Cat. #319005-CL) at 37° C. A single strand DNA (ssDNA) 23mer (P-8) which has not capacity to form G-quadruplex was used as a control. About 8 μmol stock solution of aptamers and ssDNA control (˜1.2 O.D.U) was lyophilized to dryness and then incubated with 300 μl 10% FBS at 37° C. At 0, 0.25, 0.5, 1, 2, 6 and 24 h, 50 μl of samples were collected and stored at −20° C. for at least 20 min. The samples were lyophilized to dryness and added 10 μl gel loading buffer and 10 μl autoclaved water. 10 μl of the mixture was used for polyacrylamide gel electrophoresis (PAGE) which was carried out at room temperature using 20% polyacrylamide gel in 0.5×TBE buffer (Tris-borate-EDTA). Degradation pattern on gels was visualized by Stains-All (Bio-Rad) according to manufacturer's protocol. The solution was made of 1-Ethyl-2-[3-(1-ethylnaphtho[1,2-d]thiazolin-2-ylidene)-2-methylpropenyl]naphthol[2-d]thiazolium bromide (FIGS. 6a, 6b and 6c). The data shows that nuclease stability can be dependent on one or both the position and number of FANA residues within the oligonucleotide backbone, and that the FANA residues confer significant stability against hydrolytic nucleases. For example, the nuclease stability of APT-F13 and F14 is enhanced over the native APT-35 structure by a factor of 4-7.

### EXAMPLE 5

### 5′-End Labeling of Synthetic Oligonucleotides and Filter Binding Assay

Aptamers were radioactively labeled at the 5′-hydroxyl terminus with a radioactive phosphorous probe and the enzyme T4 polynucleotide kinase (T4 PNK) according to the manufacture's specifications (MBI Fermentas Life Sciences, Burlington, ON). Incorporation of the ³²P label was accomplished in reaction mixtures consisting of DNA aptamers substrate (100 pmol), 2 μl 10×reaction buffer (Buffer A for forward reaction: 500 mM Tris-HCl, pH 7.6 at 25° C., 100 mM MgCl₂, 50 mM DTT, 1 mM spermidine and 1 mM EDTA), 1 μl T4 PNK enzyme solution (10 U/1 μl in a solution of 20 mM Tris-HCl, pH 7.5, 25 mM KCl, 0.1 mM EDTA, 2 mM DTT and 50% glycerol), 6 μl [γ-³²P]-ATP solution (6000 Ci/mmol, 10 mCi/ml; Amersham Biosciences, Inc.) and autoclaved sterile water to a final volume of 20 μl. The reaction mixture was incubated for about 45-60 min at 37° C., followed by a second incubation for 10 min at 95° C. to heat denature and deactivate the kinase enzyme. The solution was purified according to a standard protocol [Carriero, S, and Damha, M. J. (2003) Nucleic Acids Res. 31: 6157-6167] and the isolated yield of ³²P-5′-DNA following gel extraction averages 50%. The pure labeled samples were kept at −20° C. for future use.

Nitrocellulose filter binding is to confirm if there is binding between selected aptamers and thrombin. Constant amount of labeled aptamers (1.25 μmol) were heated to 95° C. for 5 minutes in the binding buffer (Tris-Ac, pH 7.4, 140 mM NaCl, 5 mM KCl, 1 mM CaCl₂, 1 mM MgCl₂) and immediately set on ice for 5 minutes before binding to increasing concentrations of thrombin protease (Amersham Biosciences, Inc.) ranging from 6-1380 nM in the binding buffer at 37° C. in a final volume of 20 μl for 30 minutes. Mixtures were filtered through a nitrocellulose filter (13 mm Millipore, HAWP, 0.45 μm) pre-wetted with binding buffer in a Millipore filter binding apparatus, and immediately rinsed with 600 μl ice cold washing buffer (Tris-Ac, pH 7.4, 140 mM NaCl, 5 mM KCl, 1 mM CaCl₂, 1 mM MgCl₂, 1% sodium pyrophosphate (w/v)), then the filter was air dried and the bound aptamer quantified by scintillation counting. The binding percentage (%) was calculated by the subtraction of counts in the miscrotube and background. K_{d} could be roughly determined by least squares f it of the data points to a binding equation that assumes a simple bimolecular RNA-thrombin interaction. The binding curves for various aptamers, including controls, are shown in FIGS. 7a and 7b, whereas binding data are given on Table 1. The data shows that binding of two (APT-F13 and F14) of the aptamers tested is quantitative and improved over the native DNA aptamer AP35. These compounds also adopt the required G-tetrad structure recognized by thrombin (“Type II” structure), as assessed by CD spectroscopy. Furthermore, the nuclease stability of APT-F13 and F14 is enhanced over APT-35 by a factor of 4-7.

### EXAMPLE 6

### Circular Dichroism (CD) Spectroscopy and Thermal Denaturation Studies Reveals G-Tetrad Formation and Stabilization by Arabinose-modified Oligonucleotides (dTTGGGGTT and dGGGGTTTTGGGG)

UV thermal denaturation data were obtained on a Varian CARY 1 spectrophotometer equipped with a Peltier temperature controller. dT₂G₄T₂ and related sequences (PG17-24) were dissolved in phosphate buffered saline (PBS buffer, pH 7.2), 137 mM NaCl, 2.7 mM KCl, 1.5 mM KH₂PO₄, 8 mM Na₂HPO₄ at a final concentration of 20 μM. dG₄T₄G₄ and related sequences (PG25-28) were dissolved in 10 mM sodium phosphate buffer, pH 7, 0.1 mM EDTA, and 200 mM NaCl; at a final concentration of 100 μM. All samples were annealed at 95° C. for 5 minutes, naturally cooled down to room temperature and refrigerated (4° C.) overnight before measurements. The annealed samples were transferred to pre-chilled Hellma QS-1.000 (Cat #114) quartz celled, sealed with a Teflon-wrapped stopper and degassed by placing them in an ultrasound bath for 1 min. Extinction coefficients were obtained from the following internet site (http://www. idtdna.com/analyzer/Applications/OligoAnalyzer/) and FANA modified sequences were assumed to have the same extinction coefficient as the regular DNA sequence. Denaturation curves were acquired at 260 nm for dT₂G₄T₂ and related sequences (PG17-24), 295 nm for dG₄T₄G₄ and related sequences (PG25-28) at a heating/cooling rate of 0.5° C./min starting from 20° C. to 90° C. (for PG17-24) or 40° C. to 98° C. (for PG25-28). The data were analyzed with the software provided by Varian Canada and converted to Microsoft Excel (Table 2).

CD spectra (220-320 nm) were collected on a Jasco J-710 spectropolarimeter at a rate of 100 nm/min using fused quartz cells (Hellma, 165-QS). Measurements were carried out in either PBS buffer for dT₂G₄T₂ and related sequences PG17-24 (pH 7.2, 137 mM NaCl, 2.7 mM KCl, 1.5 mM KH₂PO₄, 8 mM Na₂HPO₄) at a final concentration of 20 μM, or in sodium phosphate buffer for dG₄T₄G₄ and related sequences PG25-28 (10 mM sodium phosphate buffer, pH 7, 0.1 mM EDTA, and 200 mM NaCl) at a final concentration of 100 μM. Temperature was controlled by an internal circulating bath (VWR Scientific) at constant temperature (20° C.). The data were processed on a PC computer using J-700 Windows software supplied by the manufacturer (JASCO, Inc.). To facilitate comparisons, the CD spectra were background subtracted, smoothed and were corrected for concentration so that molar ellipticities could be obtained.

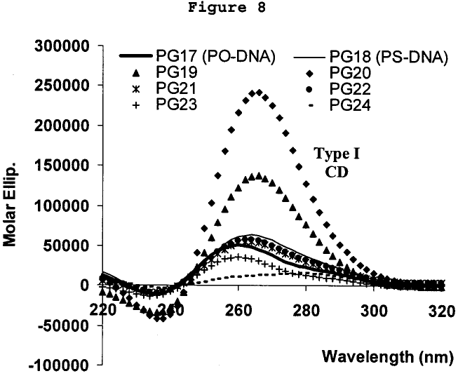

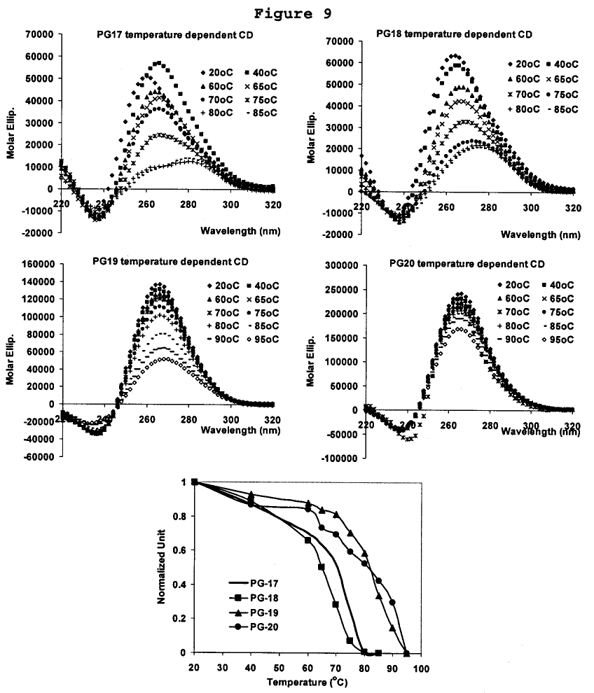

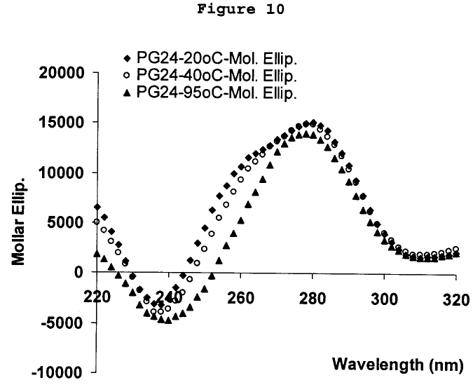

These above experiments assess the impact of the FANA modification on the G-quartet structures. The oligomer dT₂G₄T₂, is known to adopt a structure in which all of the G residues are in the anti conformation. As a result, a “Type I” signature characterizes this aptamer (Table 2 and FIGS. 8 & 9). Tₘ profiles were obtained by plotting the maximum molar ellipticities versus temperature (FIG. 10). Compared with the controls PG17 (PO-DNA) and PG18 (PS-DNA), FANA modified sequences PG19 and PG20 showed much increased molar ellipticities indicating a better guanine-guanine interactions within the G-tetrad structure. Tₘ profiles (FIG. 9) obtained by temperature-dependent CD spectra, clearly confirmed the significant thermal stabilization provided by the FANA-G units (PG-18, 19 & 20; Table 2).

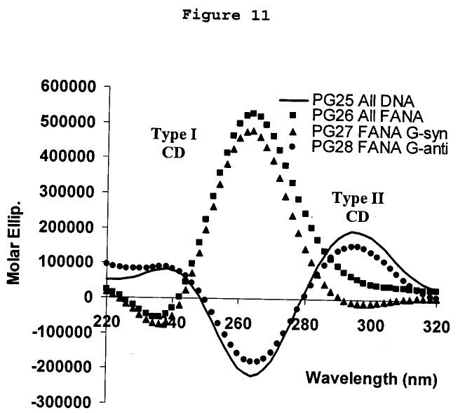

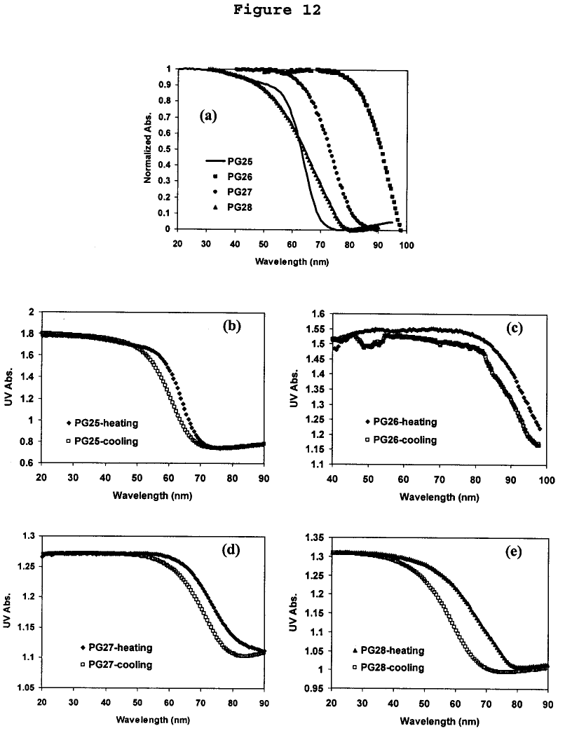

The same principles applied to the telomeric series derived from dG₄T₄G₄ (Table 2, and FIGS. 11 & 12). When the dG (anti) residues were replaced by FANA G residues, a modest thermal stabilization of the G-tetrad structure resulted without disruption of the 3-dimensional structure (Table 2, and FIGS. 11 & 12). When the dG (syn) residues were replaced, a remarkable increase in thermal stabilization of the G-tetrad structure resulted (up to 25 degrees; Table 2), with a concomitant type II-to-type I conformational change induced by the FANA G(anti) residues (FIG. 11).

All references cited are incorporated by reference herein. Although preferred embodiments of the invention have been described herein, it will be understood by those skilled in the art that variations may be made thereto without departing from the spirit of the invention or the scope of the appended claims.

TABLE 1
Sequences, CD and Tₘ data of Oligonucleotides

| | | | | | | | |
|---|---|---|---|---|---|---|---|
| | SEQ | | | | | | |
| | ID | | | Tₘ | Type II | Kd | t_{1/2} |
| Name | No. | Type | Sequence^{a} | (° C.)^{b} | CD^{c} | (nM)^{d} | (h)^{e} |

| | | | | | | | |
|---|---|---|---|---|---|---|---|
| AP32 | 1 | All FANA | 5′-GG-TT-GG-TGT-GG-TT- | 54.1 | − | 500 | >24 |
| | | | GG-3′ | | | | |
| AP33 | 2 | G-FANA/ | 5′-GG-tt-GG-tgt-GG-tt- | 50.2 | − | 450 | 4.0 |
| | | | GG-3′ | | | | |
| AP34 | 3 | G-DNA/ | 5′-gg-TT-gg-TGT-gg-TT- | 56.3 | + | 280 | 5.9 |
| | | | gg-3′ | | | | |
| AP35 | 4 | All DNA | 5′-gg-tt-gg-tgt-gg-tt- | 47.4 | + | 210 | 0.5 |
| | | | gg-3′ | | | | |
| APT- | 5 | FANA | 5′-gG-tt-gG-tgt-gG-tt- | 53.3 | + | >700 | 4.8 |
| F1 | | G-anti | gG-3′ | | | | |
| APT- | 6 | FANA | 5′-Gg-tt-Gg-tgt-Gg-tt- | 45.4 | − | >700 | 0.8 |
| F2 | | G-syn | Gg-3′ | | | | |
| APT- | 7 | FANA | 5′-gG-TT-gG-TGT-gG-TT- | 61.6 | + | 500 | 9.4 |
| F3 | | G-anti & | gG-3′ | | | | |
| | | T-loop | | | | | |
| APT- | 8 | FANA | 5′-Gg-TT-Gg-TGT-Gg-TT- | 48.5 | − | >700 | 0.6 |
| F4 | | G-syn & | Gg-3′ | | | | |
| | | T-loop | | | | | |
| APT- | 9 | FANA | 5′-gg-TT-gg-TgT-gg-TT- | 57.1 | + | 300 | 2.8 |
| F9 | | T-loop | gg-3′ | | | | |
| APT- | 10 | FANA | 5′-gg-tT-gg-TgT-gg-TT- | 51.2 | + | 250 | 2.7 |
| F10 | | T-loop | gg-3′ | | | | |
| APT- | 11 | FANA | 5′-gg-TT-gg-TgT-gg-Tt- | 56.6 | + | 370 | 5.1 |
| F11 | | T-loop | gg-3′ | | | | |
| APT- | 12 | FANA | 5′-gg-TT-gg-tgt-gg-TT- | 59.1 | + | 310 | 3.5 |
| F12 | | T-loop | gg-3′ | | | | |
| APT- | 13 | FANA | 5′-gg-tt-gg-TgT-gg-TT- | 51.0 | + | 58 | 3.4 |
| F13 | | T-loop | gg-3′ | | | | |
| APT- | 14 | FANA | 5′-gg-TT-gg-TgT-gg-tt- | 50.6 | + | 40 | 2 |
| F14 | | T-loop | gg-3′ | | | | |
| P-8 | 15 | ssDNA | 5′-gtc tct tgt gtg act | NA | NA | NC | 0.5 |
| | | control | ctg gta ac-3′ | | | | |
| H1 | 16 | Hairpin | 5′-GGAC (UUCG) GUCC-3′ | NA | NA | NC | NA |
| | | control | | | | | |

^{a}Capital and bold letter: FANA; Small letter: dna; Capital: RNA
^{b}NA: not applicable
“Type II” CD refers to a CD spectrum with positive band at ~295 nm and a negative band at ~260 nm, which indicates a alternative G-anti and G-syn conformation in the G-quartet. CD was measured in a buffer consisting of 10 mM Tris, 25 mM KCl, pH 6.8.
^{d}Kd was roughly estimated from the thrombin concentration (nM) necessary to achieve 50% of the maximum binding to the aptamer; NC: not calculated.

TABLE 2

Sequences, CD and Type Tₘ data of oligonucleotides

| | | | | | |
|---|---|---|---|---|---|
| | Seq. | | | | |
| | ID | | | | Tₘ (ΔTₘ) |
| Code | NO. | Type | Sequence^{a} | CD Type^{a,b} | (° C.)^{d,e} |

dTTGGGGTT and related sequences

| | | | | | |
|---|---|---|---|---|---|
| PG17 | 17 | All PO- | 5′-PO-tt-gggg-tt-3′ | I | 66 |
| | | DNA | | | |
| PG18 | 18 | All PS- | 5′-PS-tt-gggg-tt-3′ | I | 73.5^{f} |
| | | DNA | | | 74 (+8) |
| PG19 | 19 | All PS- | 5′-PS-TT-GGGG-TT-3′ | I | 83 (+17) |
| | | FANA | | | |
| PG20 | 20 | All FANA- | 5′-PS-tt-GGGG-tt-3′ | I | 87 (+21) |
| | | G | | | |
| PG21 | 21 | All FANA- | 5′-PS-TT-gggg-TT-3′ | I | n.d.^{g} |
| | | T | | | |
| PG22 | 22 | 2x FANA-G | 5′-PS-tt-GgGg-tt-3′ | I | n.d. |
| PG23 | 23 | 2x FANA-G | 5′-PS-tt-gGgG-tt-3′ | I | n.d. |
| PG24 | 24 | 2x FANA-G | 5′-PS-tt-GggG-tt-3′ | I | n.d. |

dGGGGTTTTGGGG and related sequences

| | | | | | |
|---|---|---|---|---|---|
| PG25 | 25 | All PO- | 5′-PO-gggg-tttt-gggg-3′ | II | 65^{h} |
| | | DNA | | | 64.4 |
| PG26 | 26 | All PO- | 5′-PO-GGGG-TTTT-GGGG-3′ | I | ~90 (+25.6) |
| | | FANA | | | |
| PG27 | 27 | G-syn | 5′-PO-GgGg-tttt-GgGg-3′ | I | 72.5 (+8.1) |
| PG28 | 28 | G-anti | 5′-PO-gGgG-tttt-gGgG-3′ | II | 66.2 (+1.8) |

^{a}Small letter: dna; Capital and bold letter: FANA; PO: phosphate linkage; PS: phosphorothioate linkage.
^{b}CD type I refers to a positive CD band at ~265 nm and a negative band at ~240; CD type II refers to a positive band at ~295 nm and a negative band at ~260 nm [Williamson, J.R. (1994) G-Quartet Structures in Telomeric DNA. Annu. Rev. Biophys. Biomol. Struct. 23: 703-730].
^{c}dT₂G₄T₂ and related sequences (PG17-24): phosphate buffered saline (PBS buffer, pH 7.2 at 25° C.), 137 mM NaCl, 2.7 mM KCl, 1.5 mM KH₂PO₄, 8 mM Na₂HPO₄; strand concentration: 20 μM for both CD and Tₘ experiments; CD was conducted at 260 nm wavelength. dG₄T₄G₄ and related sequences (PG25-28): 10 mM sodium phosphate buffer, 0.1 mM EDTA, pH 7 and 200 mM NaCl; strand concentration: 10 μM.
^{d}dT₂G₄T₂ and related sequences (PG17-24): phosphate buffered saline (PBS buffer, pH 7.2 at 25° C.), 137 mM NaCl, 2.7 mM KCl, 1.5 mM KH₂PO₄, 8 mM Na₂HPO₄; strand concentration: 20 μM; Tₘ measurements were conducted at 260 nm wavelength. dG₄T₄G₄ and related sequences (PG25-28): 10 mM sodium phosphate buffer, 0.1 mM EDTA, pH 7 and 200 mM NaCl; strand concentration: 100 μM; Tₘ measurements were conducted at 295 nm wavelength.
^{e}ΔTₘ (° C.) is the Tₘ change of PG17-24 or PG26-27 relative to the control PG17 or PG25, respectively.
^{f}ΔTₘ from the reference: Wyatt, J. R., P. W. Davis, et al. (1996) Kinetics of G-quartet-mediated tetramer formation. Biochemistry 35: 8002-8008.
^{g}n.d.: not determined
^{h}Tₘ from the reference: Lu, M., Q. Guo, et al. (1993) Biochemistry 32: 598-601.

1. An aptamer capable of forming a G-tetrad comprising at least one arabinose modified nucleotide.

2. The aptamer of claim 1, wherein the arabinose modified nucleotide has a 2′ substituent selected from the group consisting of fluorine, hydroxyl, amino, azido, alkyl, alkoxy, and alkoxyalkyl groups.

3. The aptamer of claim 2, wherein the alkyl group is selected from the group consisting of methyl, ethyl, propyl, butyl, and functionalized alkyl groups such as ethylamino, propylamino and butylamino groups, the alkoxy group is selected from the group consisting of methoxy, ethoxy, proproxy and functionalized alkoxy groups such as —O(CH₂)_{q}—R, where q=2-4 and —R is a —NH₂, —OCH₃, or —OCH₂CH₃ group and the alkoxyalkyl group is selected from the group consisting of methoxyethyl, and ethoxyethyl.

4. The aptamer of claim 3, wherein the functionalized alkyl group is selected from the group consisting of ethylamino, propylamino and butylamino group and the functionalized alkoxy group is selected from the group consisting of —O(CH₂)_{q}—R, where q=2-4 and —R is a —NH₂, -0 CH₃, or —OCH₂CH₃ group.

5. The aptamer of claim 2, wherein at least one arabinose modified nucleotide is 2′-deoxy-2′-fluoroarabinonucleotide (FANA).

6. The aptamer of claim 1, wherein at least one arabinose modified nucleotide is in a loop of the G-Tetrad.

7. The aptamer of claim 1, wherein at least one arabinose modified nucleotide is a guanosine residue of the G-tetrad.

8. The aptamer of claim 1, wherein aptamer selectively binds thrombin.

9. The aptamer of claim 8, wherein the aptamer comprises the nucleotide sequence dGGTTGGTGTGGTTGG.

10. The aptamer of claim 1 having a sequence selected from group consisting of SEQ ID NOS. 1-3 and 4-14.

11. The aptamer of claim 1, wherein the aptamer selectively binds HIV gp120.

12. The aptamer of claim 11, wherein the aptamer comprises the nucleotide sequence dTTGGGGTT.

13. The aptamer of claim 1 having a sequence selected from group consisting of SEQ ID NOS. 19-24.

14. The aptamer of claim 1, wherein the aptamer is a dG4T4 repeat.

15. The aptamer of claim 14, wherein the aptamer is any one of dG4T4G4, dG4T4G4T4G4 and dG4T4G4T4G4T4G4.

16. The aptamer of claim 1 having a sequence selected from the group consisting of SEQ ID NOS. 26-28.

17. The aptamer of claim 1 having a sugar phosphate backbone.

18. The aptamer of claim 1 wherein the aptamer is a chimera of 2′-deoxyribonucleotide (DNA) and 2′-deoxy-2′-fluoroarabinonucleotide (FANA) nucleotides.

19. The aptamer of claim 1, containing at least one internucleotide linkage selected from the group consisting of phosphodiester, phosphotriester, phosphorothioate, methylphosphonate, boranophosphate and any combination thereof.

20. A method for increasing at least one of nuclease stability or selective binding of an aptamer comprising replacing at least one nucleotide of the aptamer with an arabinose modified nucleotide.

21. The method of claim 20, wherein the arabinose modified nucleotide is 2′-deoxy-2′-fluoroarabinonucleotide (FANA).

22. The method of claim 20, wherein the aptamer forms a G-tetrad.

23. The method of claim 22, wherein at least one nucleotide being replaced is in a loop of the G-tetrad.

24. The method of claim 22, wherein at least one nucleotide being replaced is a guanosine residue of the G-tetrad.

25. The method of claim 20 wherein the aptamer selectively binds thrombin and has the nucleotide sequence dGGTTGGTGTGGTTGG.

26. The method of claim 20 wherein the aptamer selectively bind HIV gp120 and has the nucleotide sequence dTTGGGGTT.

27. The method of claim 20 wherein the aptamer is a dG4T4 repeat and is any one of dG4T4G4, dG4T4G4T4G4 and dG4T4G4T4G4T4G4.

28. A pharmaceutical composition comprising the aptamer of claim 1 and a pharmaceutically acceptable carrier.

29. A pharmaceutical composition comprising the aptamer of claim 8 and a pharmaceutically acceptable carrier.

30. A pharmaceutical composition comprising the aptamer of claim 11 and a pharmaceutically acceptable carrier.

31. A pharmaceutical composition comprising the aptamer of claim 14 and along with a pharmaceutically acceptable carrier.

32. (canceled)

33. A method of inhibiting thrombin in a patient in need thereof, comprising administering a therapeutically effective amount of the composition of claim 29.

34. A commercial package comprising the composition of claim 29 together with instructions for its use for inhibiting thrombin and increasing blood clotting times.

35. (canceled)

36. A method of treating or preventing HIV infection in a patient in need thereof, comprising administering a therapeutically effective amount of the composition of claim 30.

37. A commercial package comprising the composition of claim 30 together with instructions for its use for treating or preventing HIV infection.

38. (canceled)

39. A method of treating or preventing cancer in a patient in need thereof, comprising administering a therapeutically effective amount of the composition of claim 31.

40. A commercial package comprising the composition of claim 31 together with instructions for its use for treating or preventing cancer.


SEQUENCE LISTING (20 sequences)

SEQ ID NO: 1 - DNA, 15 bp, Artificial Sequence
  synthetic oligonucleotide 1
nnnnnnnnnnnnnnn

SEQ ID NO: 2 - DNA, 15 bp, Artificial Sequence
  synthetic oligonucleotide 2
nnttnntgtnnttnn

SEQ ID NO: 3 - DNA, 15 bp, Artificial Sequence
  synthetic oligonucleotide 3
ggnnggnnnggnngg

SEQ ID NO: 4 - DNA, 15 bp, Artificial Sequence
  synthetic oligonucleotide 4
ggttggtgtggttgg

SEQ ID NO: 5 - DNA, 15 bp, Artificial Sequence
  synthetic oligonucleotide 5
gnttgntgtgnttgn

SEQ ID NO: 6 - DNA, 15 bp, Artificial Sequence
  synthetic oligonucleotide 6
ngttngtgtngttng

SEQ ID NO: 7 - DNA, 15 bp, Artificial Sequence
  synthetic oligonucleotide 7
gnnngnnnngnnngn

SEQ ID NO: 8 - DNA, 15 bp, Artificial Sequence
  synthetic oligonucleotide 8
ngnnngnnnngnnng

SEQ ID NO: 9 - DNA, 15 bp, Artificial Sequence
  synthetic oligonucleotide 9
ggnnggngnggnngg

SEQ ID NO: 10 - DNA, 15 bp, Artificial Sequence
  synthetic oligonucleotide 10
ggtnggngnggnngg

SEQ ID NO: 11 - DNA, 15 bp, Artificial Sequence
  synthetic oligonucleotide 11
ggnnggngnggntgg

SEQ ID NO: 12 - DNA, 15 bp, Artificial Sequence
  synthetic oligonucleotide 12
ggnnggtgtggnngg

SEQ ID NO: 13 - DNA, 15 bp, Artificial Sequence
  synthetic oligonucleotide 13
ggttggngnggnngg

SEQ ID NO: 14 - DNA, 15 bp, Artificial Sequence
  synthetic oligonucleotide 14
ggnnggngnggttgg

SEQ ID NO: 15 - DNA, 23 bp, Artificial Sequence
  synthetic oligonucleotide 15
gtctcttgtgtgactctggtaac

SEQ ID NO: 16 - RNA, 12 nt, Artificial Sequence
  synthetic oligonucleotide 16
ggacuucggucc

SEQ ID NO: 17 - DNA, 12 bp, Artificial Sequence
  synthetic oligonucleotide 25
ggggttttgggg

SEQ ID NO: 18 - DNA, 12 bp, Artificial Sequence
  synthetic oligonucleotide 26
nnnnnnnnnnnn

SEQ ID NO: 19 - DNA, 12 bp, Artificial Sequence
  synthetic oligonucleotide 27
ngngttttngng

SEQ ID NO: 20 - DNA, 12 bp, Artificial Sequence
  synthetic oligonucleotide 28
gngnttttgngn